(19) United States
(12) Patent Application Publication
(10) Pub. No.: US 2016/0015451 A1
(43) Pub. Date: Jan. 21, 2016

(54) SYSTEMS AND METHODS FOR TREATING TISSUE WITH RADIOFREQUENCY ENERGY

(71) Applicant: Mederi Therapeutics, Inc., Norwalk, CT (US)

(72) Inventor: Oleg Shikhman, Trumbull, CT (US)

(73) Assignee: Mederi Therapeutics, Inc., Norwalk, CT (US)

(21) Appl. No.: 14/866,664

(22) Filed: Sep. 25, 2015

Related U.S. Application Data
(63) Continuation-in-part of application No. 14/708,210, filed on May 9, 2015.
Application No. 14/708,210 is a continuation-in-part of application No. 13/867,042, filed on Apr. 20, 2013.
Application No. 13/867,042 is a continuation-in-part of application No. 12/924,155, filed on Sep. 22, 2010, now abandoned.
(60) Provisional application No. 62/065,658, filed on Oct. 18, 2014.
(60) Provisional application No. 62/009,223, filed on Jun. 7, 2014.
(60) Provisional application No. 61/664,960, filed on Jun. 27, 2012.
(60) Provisional application No. 61/277,260, filed on Sep. 22, 2009.

(51) Int. Cl. A61B 18/14

(52) U.S. Cl. CPC A61B 18/1492; A61B 2018/1475; A61B 2018/1425; A61B 2218/007; A61B 2018/00571

(57) ABSTRACT

A flexible sleeve mountable to an endoscope having a proximal portion, a distal portion, a first channel dimensioned to receive an endoscope, and a second channel dimensioned to receive an electrode. The electrode is slidable within the second channel from a retracted position wherein a tip of the electrode is unexposed from the sleeve and an extended position wherein the tip of the electrode is exposed from the sleeve and curves toward target tissue.

### RELATED APPLICATIONS

This application claims priority from provisional application Ser. No. 62/065,658, filed Oct. 18, 2014, and is a continuation-in-part of application Ser. No. 14/708,210, filed May 9, 2015, which claims the benefit of provisional application Ser. No. 62/009,223, filed Jun. 7, 2014, and is a continuation-in-part of application Ser. No. 13/867,042, filed Apr. 20, 2013, which claims the benefit of provisional application Ser. No. 61/664,960, filed Jun. 27, 2012, and is a continuation-in-part of application Ser. No. 12/924,155, filed Sep. 22, 2010, which claims the benefit of provisional application Ser. No. 61/277,260 filed Sep. 22, 2009. The entire contents of each of these applications are incorporated herein by reference.

### FIELD OF THE INVENTION

In a general sense, the invention is directed to systems and methods for treating interior tissue regions of the body. More specifically, the invention is directed to a device mountable to an endoscope for treating dysfunction in body sphincters and adjoining tissue by applying radiofrequency energy to tissue to create tissue lesions.

### BACKGROUND OF THE INVENTION

The gastrointestinal (GI) tract, also called the alimentary canal, is a long tube through which food is taken into the body and digested. The alimentary canal begins at the mouth, and includes the pharynx, esophagus, stomach, small and large intestines, and rectum. In human beings, this passage is about 30 feet (9 meters) long.

Small, ring-like muscles, called sphincters, surround portions of the alimentary canal. In a healthy person, these muscles contract or tighten in a coordinated fashion during eating and the ensuing digestive process, to temporarily close off one region of the alimentary canal from another region of the alimentary canal.

For example, a muscular ring called the lower esophageal sphincter (or LES) surrounds the opening between the esophagus and the stomach. Normally, the lower esophageal sphincter maintains a high-pressure zone between fifteen and thirty mm Hg above intragastric pressures inside the stomach.

In the rectum, two muscular rings, called the internal and external sphincter muscles, normally keep fecal material from leaving the anal canal. The external sphincter muscle is a voluntary muscle, and the internal sphincter muscle is an involuntary muscle. Together, by voluntary and involuntary action, these muscles normally contract to keep fecal material in the anal canal.

Dysfunction of a sphincter in the body can lead to internal damage or disease, discomfort, or otherwise adversely affect the quality of life. For example, if the lower esophageal sphincter fails to function properly, stomach acid may rise back into the esophagus. Heartburn or other disease symptoms, including damage to the esophagus, can occur. Gastrointestinal reflux disease (GERD) is a common disorder, characterized by spontaneous relaxation of the lower esophageal sphincter.

Damage to the external or internal sphincter muscles in the rectum can cause these sphincters to dysfunction or otherwise lose their tone, such that they can no longer sustain the essential fecal holding action. Fecal incontinence results, as fecal material can descend through the anal canal without warning, stimulating the sudden urge to defecate. The physical effects of fecal incontinence (i.e., the loss of normal control of the bowels and gas, liquid, and solid stool leakage from the rectum at unexpected times) can also cause embarrassment, shame, and a loss of confidence, and can further lead to mental depression.

In certain surgical systems, radiofrequency energy is applied to tissue at different tissue levels to create multiple tissue lesions. Application of such energy requires continuous monitoring of certain tissue and/or device parameters to ensure that the tissue is not heated to such extent that damaging burning of tissue occurs. Thus, these systems monitor tissue temperature and/or device electrode temperature and provide safety features to cut off energy flow if the tissue temperature rises too high. However, with the application of radiofrequency energy, there is a fine point in which tissue is treated to form lesions and beneficially alter structure of the tissue, e.g., alter the structure of the sphincter muscle, while not being ablated.

Ablation of tissue can be generally defined as a removal of a part of tissue. Radiofrequency energy to ablate tissue has been used for various tumor treatments, destroying tissue and creating tissue necrosis. However, avoiding tissue ablation may be beneficial in treating the gastrointestinal tract in the foregoing or other procedures. Therefore, it would be advantageous to provide a system of applying radiofrequency energy to tissue at a power setting and time duration which causes thermal effect to tissue to create tissue lesions along a series of tissue levels but avoids ablation or burning of tissue.

However, in avoiding tissue ablation, care needs to be taken to ensure that tissue is not undertreated. In other words, in attempts to prevent overheating of tissue which causes ablation, the system needs to conversely ensure that tissue is not under-heated and thus not therapeutically treated. Therefore, the need exists for a system that applies radiofrequency energy to tissue between these two energy levels.

It would also be advantageous to provide a system for applying radiofrequency energy to tissue which could be mounted to an endoscope, thereby providing an inexpensive easily adaptable system for treating tissue with radiofrequency energy.

### SUMMARY OF THE INVENTION

This system in one aspect provides flexible sleeve with one or more electrodes and a channel to receive an endoscope such that the endoscope is inserted within the sleeve and one or more electrodes are deployed from the sleeve, with electrode tissue penetration limited to control depth of insertion to provide a consistent predetermined depth of penetration at the lesion levels.

In accordance with another aspect, the present invention provides a device for applying radiofrequency energy for sphincter treatment comprising a flexible sleeve having a lumen to removably receive an endoscope and an external channel to movably receive an electrode.

In accordance with another aspect, the present invention provides a flexible sleeve fitted over a substantial length of an endoscope and an electrode movable within the sleeve to an exposed position to apply energy to tissue.

In accordance with another aspect, a flexible sleeve mountable to an endoscope is provided, the sleeve comprising a proximal portion, a distal portion, a first channel dimensioned to receive an endoscope, and a second channel dimensioned to receive an electrode. The electrode is slidable within the second channel from a retracted position wherein a tip of the electrode is unexposed from the sleeve and an extended position wherein the tip of the electrode is exposed from the sleeve and curves toward target tissue.

In some embodiments, the tip of the electrode is a penetrating tip to penetrate the target tissue. In some embodiments, the flexible sleeve includes a third channel and a second electrode is slidable within the third channel from a retracted position wherein a tip of the second electrode is unexposed from the sleeve and an extended position wherein the tip of the second electrode is exposed from the sleeve and curves toward target tissue.

The sleeve can include in some embodiments an actuator operatively connected to the electrode for moving the electrode between retracted and exposed positions.

In some embodiments, a stop is positioned on the electrode to control a depth of penetration of the electrode to provide a consistent depth of penetration at the lesion levels. The sleeve can include an aspiration lumen for aspiration of particles. In some embodiments, the second channel is on an exterior surface of the sleeve.

In some embodiments, the sleeve includes an expandable member expandable to force the electrode further into tissue. In some embodiments, the expandable member is an inflatable balloon.

In accordance with another aspect, a system is provided comprising a flexible endoscope and a flexible sleeve mountable over the endoscope to apply energy to form lesions in a treatment of gastrointestinal reflux disease under visualization. The sleeve has a first channel to receive the endoscope therein and a second channel for slidably receiving an electrode, the electrode slidable within the second channel from a retracted position wherein a tip of the electrode is unexposed from the sleeve and an extended position wherein the tip of the electrode is exposed from the sleeve and curves toward target tissue. The electrode is connectable to a generator for application of energy to tissue to form lesions to tighten a sphincter.

In some embodiments, non-ablative energy is applied to the electrode. In some embodiments, the electrode includes a stop to limit penetration depth to prevent overtreatment of tissue. In some embodiments, the sleeve is placed over a substantial portion of the endoscope; in other embodiments, the sleeve is placed over only a distal portion of the endoscope.

The sleeve can include a second channel and a second electrode slidable within the second channel from a retracted position wherein a tip of the second electrode is unexposed from the sleeve and an extended position wherein the tip of the second electrode is exposed from the sleeve and curves toward target tissue, the second electrode connectable to a generator for application of energy to tissue to form lesions to tighten a sphincter.

In accordance with another aspect, a method for forming lesions in a sphincter is provided comprising the steps of:
a. providing an endoscope;
b. providing a flexible sleeve;
c. mounting the flexible sleeve over at least a distal portion of the endoscope;
d. inserting the sleeve and endoscope into a body to position adjacent target tissue;
e. extending an electrode from the sleeve to penetrate tissue; and
f. applying energy to tissue to form lesions.

In some embodiments, the step of applying energy applies non-ablative energy. In some embodiments, the electrode includes a stop and the step of extending the electrode extends the electrode until the stop prevents further penetration to thereby limit the depth of penetration to control application of energy. In some embodiments, the step of expanding an expandable member advances the electrode further into tissue.

In some embodiments, the present provides an electrosurgical system that applies radiofrequency energy to tissue to create tissue lesions at different tissue levels and alters the structure of the tissue, e.g., the sphincter muscle, without ablating or burning the tissue, while on the other hand reducing the incidence of tissue undertreatment. That is, the present invention in such embodiments provides such electrosurgical system that avoids such overheating of tissue, while at the same time limiting under-heating of tissue which does not effectively treat tissue. In striking this balance between the overheating and under heating of tissue, more reliable and consistent tissue treatment is achieved. Thus, the system keeps tissue treatment within a target zone to provide a therapeutic effect to tissue, defined as thermally heating tissue above a lower parameter wherein tissue is undertreated and below a tissue ablation threshold wherein tissue is overheated and ablated.

The device and endoscope of the present invention form in some embodiments a system enabling treating tissue with radiofrequency energy without ablating the tissue. However, the device and endoscope can also form a system used to ablate tissue if desirable in certain applications. The devices disclosed herein are designed to apply radiofrequency energy, although other forms of energy could be utilized.

### BRIEF DESCRIPTION OF THE DRAWINGS

FIG. 1 is perspective view of one embodiment of the system of the present invention shown transorally inserted;

FIG. 2 is a perspective view showing an endoscope being advanced into one embodiment of a sleeve of the present invention;

FIG. 3 is a transverse cross-sectional of the sleeve of FIG. 2 and endoscope positioned therein;

FIG. 4 is a longitudinal cross-sectional view of the system of FIG. 2, the electrode shown in the retracted position;

FIG. 5 is a view similar to FIG. 4 showing the electrode in the advanced position;

FIG. 6 is a longitudinal cross-sectional view of a distal end of an alternate embodiment of the system of the present invention, the electrode having a tissue stop and shown in the retracted position;

FIG. 7 is a view similar to FIG. 6 showing the electrode in the advanced position;

FIG. 8 is a perspective view of a distal end of an alternate embodiment of the system of the present invention, the electrodes shown in the retracted position;

FIG. 9 is a perspective view similar to FIG. 8 showing the electrodes in the advanced position;

FIG. 10 is a longitudinal cross-sectional view of the system of FIG. 8 showing the electrodes in the retracted position;

FIG. 11 is a perspective view of a distal end of an alternate embodiment of the system of the present invention, the electrodes shown in the retracted position;

FIG. 12 is a perspective view of a distal end of another alternate embodiment of the system of the present invention, the electrodes shown in the retracted position;

FIG. 13 is a perspective view of a distal end of yet another alternate embodiment of the system of the present invention, the electrode shown in the retracted position;

FIG. 14 is a longitudinal cross-sectional view of the system of FIG. 13 showing the electrode in the retracted position;

FIG. 15 is a view similar to FIG. 14 showing the electrode in the advanced position;

FIG. 16 is a longitudinal cross-sectional view of a distal end of an alternate embodiment of the system of the present invention, the electrode shown in the retracted position;

FIG. 17 is a view similar to FIG. 16 showing the electrode in the advanced position;

FIG. 18 is a longitudinal cross-sectional view of a distal end of an alternate embodiment of the system of the present invention, the electrodes shown in the retracted position;

FIG. 19 is a view similar to FIG. 18 showing the electrodes in the advanced position;

FIG. 20 is a longitudinal cross-sectional view of a distal end of an alternate embodiment of the system of the present invention, the electrode shown in the retracted position;

FIG. 21 is a view similar to FIG. 20 showing the electrode in the advanced position;

FIG. 22 is a longitudinal cross-sectional view of a distal end of an alternate embodiment of the system of the present invention, the electrode shown in the retracted position;

FIG. 23 is a view similar to FIG. 22 showing the electrode in the advanced position;

FIG. 24 is a longitudinal cross-sectional view of a distal end of another alternate embodiment of the system of the present invention, the electrodes shown in the retracted position;

FIG. 25 is a view similar to FIG. 24 showing the electrodes in the partially advanced position;

FIG. 26 is a longitudinal cross-sectional view of a distal end of an alternate embodiment of the system of the present invention, the electrode shown in the retracted position and the balloon in a deflated condition;

FIG. 27 is a view similar to FIG. 26 showing the electrode in the advanced position and the balloon in the deflated condition;

FIG. 28 is a view similar to FIG. 26 showing the electrode in the advanced position and the balloon in the inflated condition;

FIG. 29 is a longitudinal cross-sectional view of a distal end of an alternate embodiment of the system of the present invention, the electrode shown in the retracted position and the expander in a collapsed condition;

FIG. 30 is a view similar to FIG. 29 showing the electrode in the advanced position and the expander in the collapsed condition;

FIG. 31 is a view similar to FIG. 30 showing the electrode in the advanced position and the expander in the expanded condition; and

FIG. 32 illustrates one desired formation of lesions utilizing the systems of the present invention.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DESCRIPTION OF PREFERRED EMBODIMENTS

This specification discloses various systems and methods for treating dysfunction of sphincters and adjoining tissue regions in the body. The systems and methods are particularly well suited for treating these dysfunctions in the upper gastrointestinal tract, e.g., gastro-esophageal reflux disease (GERD) affecting the lower esophageal sphincter and adjacent cardia of the stomach. For this reason, the systems and methods will be described in this context. Still, it should be appreciated that the disclosed systems and methods are applicable for use in treating other dysfunctions elsewhere in the body, including dysfunctions that are not necessarily sphincter-related. For example, the various aspects of the invention have application in procedures requiring treatment of hemorrhoids, or fecal incontinence, or urinary incontinence, or restoring compliance to or otherwise tightening interior tissue or muscle regions.

The systems apply energy in a selective fashion to tissue in or adjoining the targeted sphincter region. The applied energy creates one or more lesions, or a prescribed pattern of lesions, below the surface of the targeted region. The subsurface lesions are desirably formed in a manner that preserves and protects the surface against thermal damage. Preferably, the energy is applied to the muscle layer, beyond the mucosa layer.

Natural healing of the subsurface lesions leads to a reconstruction/remodeling of the tissue which leads to beneficial changes in properties of the targeted tissue. The subsurface lesions can also result in the interruption of aberrant electrical pathways that may cause spontaneous sphincter relaxation. In any event, the treatment can restore normal closure function to the sphincter region as the application of radiofrequency energy beneficially changes the properties of the sphincter muscle wall. Such energy rejuvenates the muscle to improve muscle function.

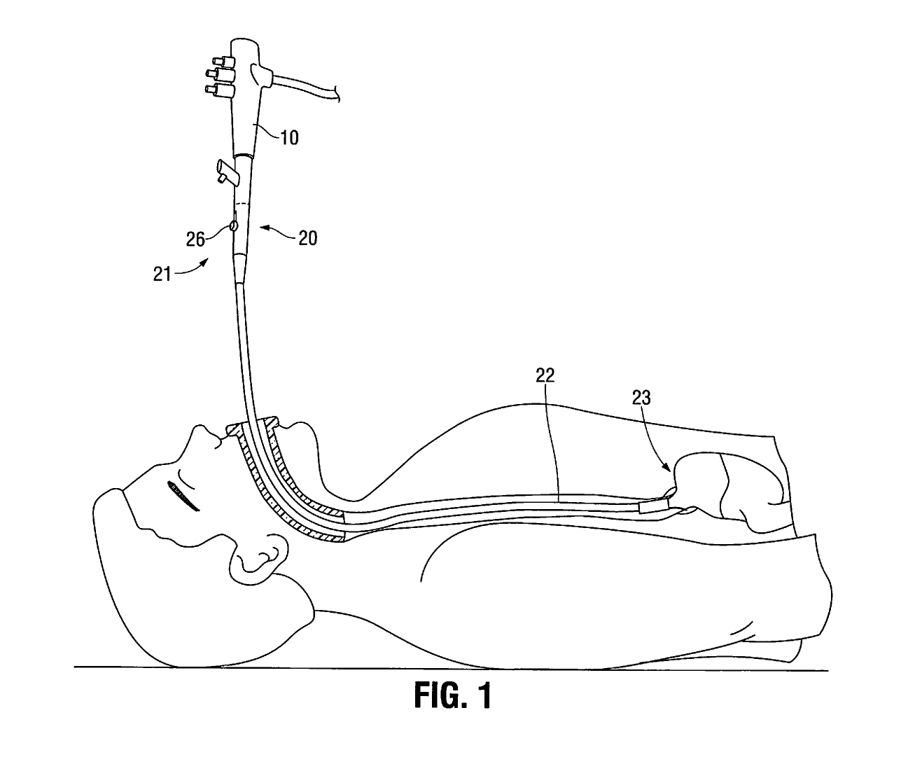

The device 20 (FIG. 1) of a first embodiment is connected to a generator (not shown) to supply the treatment energy to the electrode(s). The generator preferably supplies radio frequency energy, e.g., having a frequency in the range of about 400 kHz to about 10 mHz, although other ranges are contemplated. Other forms of energy can alternatively be applied, e.g., coherent or incoherent light; heated or cooled fluid; resistive heating; microwave; ultrasound; a tissue ablation fluid; or cryogenic fluid. The device 20 is coupled to the generator via a cable connector to convey the generated energy to the respective electrode.

The system preferably can also include certain auxiliary processing equipment such as an external fluid delivery apparatus and an external aspiration apparatus. Alternatively or in addition, the endoscope channels in the device 20 can be used for fluid delivery and/or for aspiration.

The system can also include a controller (not shown), which preferably includes a central processing unit (CPU), linked to the generator, and can be linked to the fluid delivery apparatus, and the aspiration apparatus. Alternatively, the aspiration apparatus can comprise a conventional vacuum source typically present in a physician's suite, which operates continuously, independent of the controller.

The controller governs the power levels, cycles, and duration that the radio frequency energy is distributed to the device 20 to achieve and maintain power levels appropriate to achieve the desired treatment objectives. In tandem, the controller can also govern the delivery of processing fluid and, if desired, the removal of aspirated material. Thus, the controller maintains the target tissue temperature to ensure the tissue is not overheated.

The controller can include an input/output (I/O) device (not shown) which allows the physician to input control and processing variables, to enable the controller to generate appropriate command signals. The I/O device can also receive real time processing feedback information from one or more sensors associated with the electrode or device, for processing by the controller, e.g., to govern the application of energy and the delivery of processing fluid. The I/O device can also include a graphical user interface (GUI) to graphically present processing information to the physician for viewing or analysis.

The radio frequency generator, the controller with I/O device, and the fluid delivery apparatus (e.g., for the delivery of cooling liquid) can all be integrated within a single housing. The I/O device can couple the controller to a display microprocessor. The display microprocessor can be coupled to a graphics display monitor in the housing. The controller can implement through the display microprocessor the graphical user interface, or GUI, which is displayed on the display monitor. The graphical user interface can be realized with conventional graphics software using the MS WINDOWS® application. The GUI can be implemented by showing on the monitor basic screen displays.

Turning now to the treatment devices of the present invention, in general, the device 20 of a first embodiment is a device for treating sphincter regions in the upper gastro-intestinal tract, and more particularly, the lower esophageal sphincter and adjoining cardia of the stomach to treat GERD. As noted herein, the device 20, as with the other devices described herein, can alternatively be used for other procedures and used in other areas of the patient's body. In the embodiment shown in FIG. 1, the device 20 includes an elongated flexible sleeve 22 with channels formed therein. Note that for clarity throughout the drawings not all identical components are labeled in the specific drawings.

With reference to FIGS. 1-5, wherein like reference numerals refer to like parts throughout the several views, device 20 of the first embodiment has a flexible sleeve 22 having a proximal portion 21 and a distal portion 23. Contained within the flexible sleeve 22 is a needle electrode 32 with a penetrating tip 32a. The sleeve 22 is configured to be mounted over a conventional endoscope, thereby creating a system for applying energy to tissue under visualization.

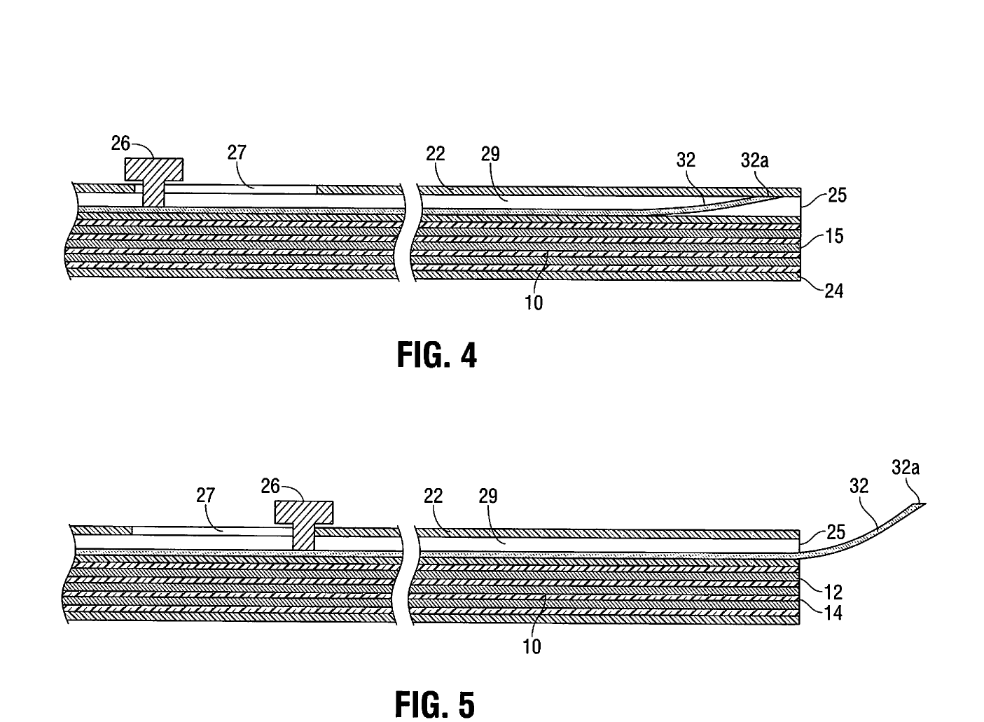

The needle electrode 32 is axially movable from a retracted position within the sleeve 22 for delivery to an advanced position protruding through distal opening 25 of sleeve 22. A plug can extend from the sleeve 22 and be electrically connected to the electrode 32 to electrically communicate with a generator to apply radiofrequency to the electrode 32 for application of energy to treat tissue. In some embodiments, this can be non-ablative energy to prevent ablation of tissue while still enabling formation of lesions. Actuator 26 on sleeve 22 is one type of actuator mechanism that can be used to advance the needle electrode 32. In this mechanism, the actuator is in the form of a slider 26 movable within longitudinal slot 27 of sleeve 22 from an initial or first position of FIG. 4 to a second advanced position of FIG. 5 to advance the electrode 32 from the sleeve 22. The slider 26 can be operatively connected to the electrode 32 either by direct attachment to the electrode 32 or alternatively by attachment to an advancer and the proximal end of the electrode 32 is attached to the distal end of the advancer such that movement of the slider 26 moves the advancer to thereby move the electrode 32. In the retracted position, the electrode 32 is less curved and preferably substantially aligned with the longitudinal axis of the sleeve 22, and when deployed from the channel or lumen 29, assumes the pre-bent, e.g., shape memorized, curved configuration.

As used herein, the terms attached or coupled or connected are not limited to direct attachment as interposing components can be used.

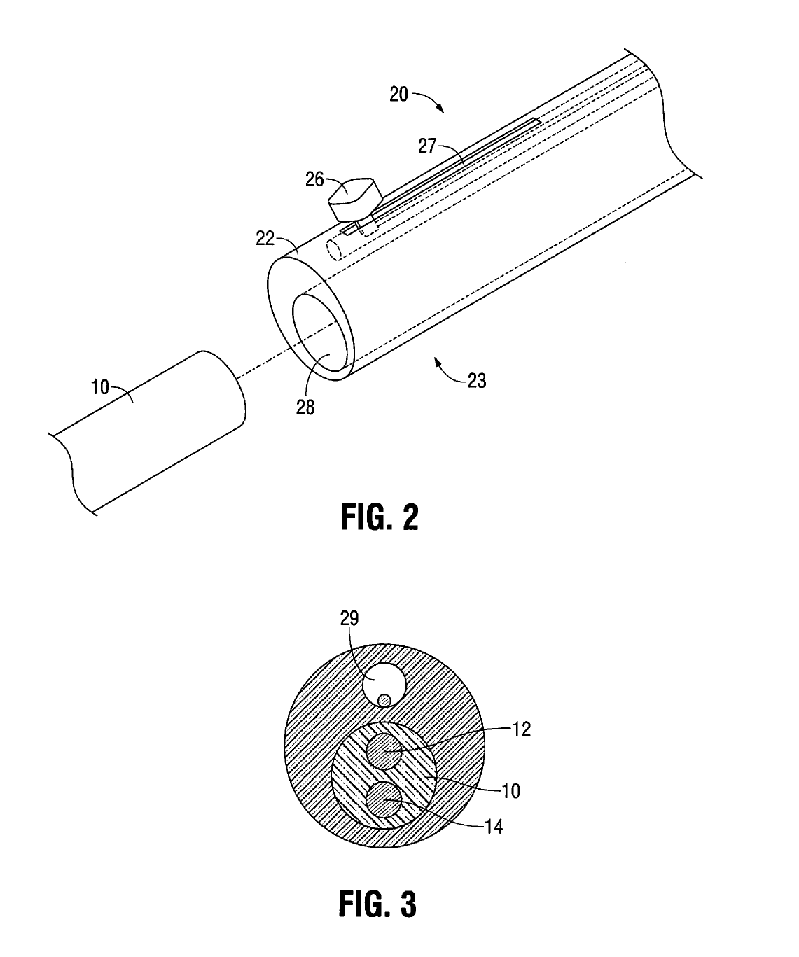

The sleeve 22 has an endoscope receiving channel or lumen 28 and an electrode receiving channel 29, as shown in FIGS. 2 and 3. As shown, the channels 28 and 29 are preferably located off-center, i.e., offset from a center longitudinal axis of the sleeve 22. Endoscope receiving channel 28 can receive a conventional endoscope. 10. The scope 10 has an imaging system 12 and a light system 14 to illuminate the surgical site. Channel 29 is configured to slidably receive the electrode 32, and such slidable movement is controlled by sliding actuator 26. Channel 29 has an open distal end (distal opening) 25 so that the distal portion of the electrode 32 can exit the channel 29 as shown in FIG. 5 and advance distal of a distal edge of the sleeve 22.

In use, prior to insertion into a patient's body, the endoscope 10 is inserted through the channel 28 of sleeve 22 (or the sleeve 22 is fitted over the endoscope 10) covering the entire length or a substantial length of the endoscope 10. The distal end 15 of the endoscope 10 preferably terminates at the distal end 24 of the sleeve 22. The endoscope 10 can be a conventional endoscope such that the sleeve 22 can be provided as a separate unit and combined with the endoscope 10 by the clinician prior to use. The combined endoscope and sleeve are inserted into the patient's body, adjacent the sphincter, and the slider 26 is slid distally from the retracted position of FIG. 4 to the advanced position of FIG. 5 (within longitudinally extending slot 27), thus advancing and exposing the electrode distal portion (and penetrating tip 32a) from within the confines of the sleeve 22. Preferably, the electrode 32 has a preformed bend so when exposed it curves in a direction away from the longitudinal axis of the sleeve 22 as shown in FIG. 5. The endoscope can be a steerable endoscope so that steering/angling of the distal end of the endoscope will also angle the sleeve 22 and electrode 32 contained therein.

The systems herein provide application of radiofrequency energy to tissue via one or more electrodes. The energy is applied via the electrode(s) to tissue at a series of axially spaced tissue levels, thereby forming tissue lesions which alters the tissue structure. Prior application of radiofrequency energy to tissue in various surgical procedures involved application of energy at certain levels and for a certain period of time with the goal to ablate the tissue. That is, the objective was to cause tissue necrosis and remove tissue.

The systems and methods of the present disclosure can in certain applications be used to treat tissue without ablating the tissue and without causing tissue necrosis, which advantageously achieves better clinical results, especially when treating the sphincter muscles of the GI tract in the specific surgical procedures disclosed herein. By applying sufficient energy to cause thermal effect to tissue, but without ablating or burning the tissue, tissue reconstruction/remodeling occurs which results in beneficial changes to tissue properties, thus beneficially treating GERD which is caused by the spontaneous relaxation of the lower esophageal sphincter and beneficially treating fecal incontinence caused by loss of tone of the sphincter muscles in the anal canal. The systems of the present disclosure rejuvenate muscle to improve muscle function. This also increases the smooth muscle/connective ratio which results in sphincter reinforcement and remodeling.

In studies performed, it was found that application of non-ablative RF energy to sphincter muscle influences the structural arrangement of smooth muscle and connective tissue contents. The increase of the smooth muscle fibers area per muscle bundles as well as the collagen and myofibroblast contents within the internal anal sphincter were found to be potentially responsible for sphincter reinforcement and remodeling. More specifically, in studies, it was found that application of non-ablative RF energy increased smooth muscle/connective tissue ratio without changes (increase) in the collagen I/III ratio. There was an increase in diameter and number of type I fibers in the external anal sphincter after non-ablative RF and higher cellular smooth muscle content in the internal anal sphincter, suggesting that sphincter remodeling by non-ablative RF energy resulted from activation and repopulation of smooth muscle cells, possibly related to phenotype switch of fibroblasts into myofibroblasts and external anal sphincter fibers. In one animal study, quantitative image analysis showed the cross-section occupied by smooth muscle within the circular muscle increased by up to 16% after non-ablative RF, without increase in collagen ratio, and external anal sphincter muscle fiber type composition showed an increase in type I/III fiber ratio from 26.2% to 34.6% after non-ablative RF, as well as a 20% increase in fiber I type diameter compared to controls.

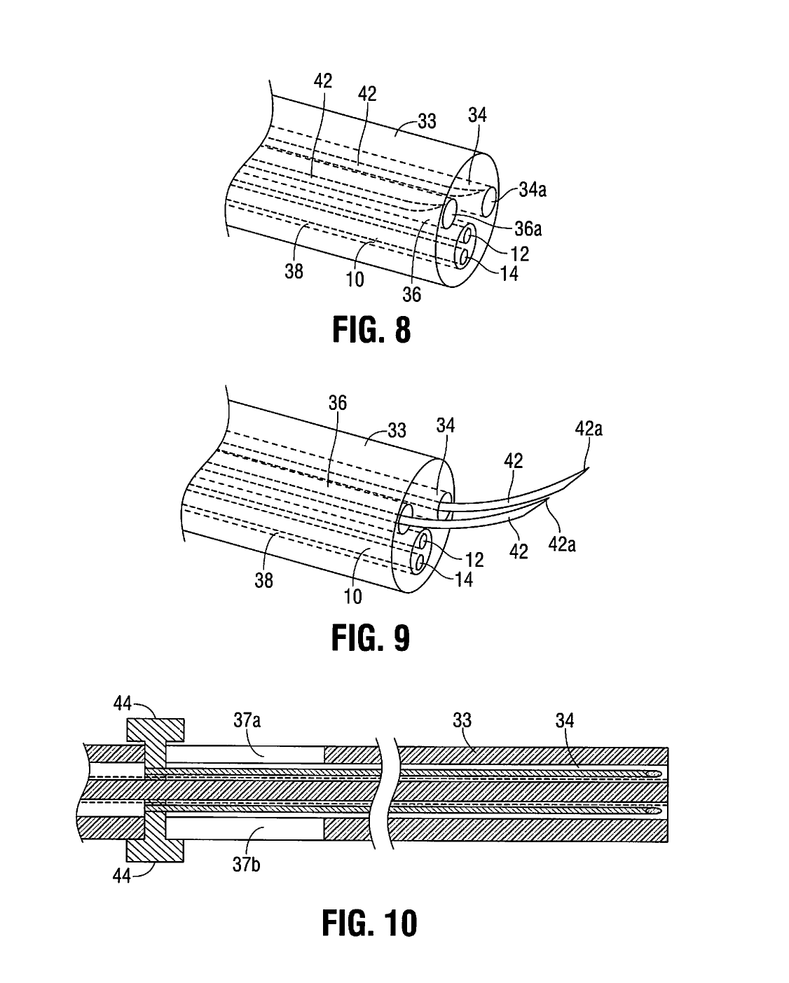

FIGS. 8-10 illustrate an alternate embodiment of the flexible sleeve. The sleeve 33 has two side by side electrode channels (or lumens) 34, 36 each configured to slidingly receive a needle electrode 42. Sleeve 33 also has a channel 38 to receive an endoscope 10 as in the embodiment of FIG. 1 and preferably extends over the entire length or a substantial length of the endoscope 10. Therefore, the embodiment of FIG. 8 differs from that of FIG. 1 in that two needle electrodes 42 are provided in separate channels of the sleeve 33 and two actuators e.g., sliders 44, are provided, each operatively connected to one of the electrodes 42. The sliders 44 are movable within a respective slot 37a, 37b of sleeve 33 from the retracted position of FIG. 8 to the advanced position of FIG. 9 to deploy the electrodes 42 from the sleeve 33. i.e., out of a respective distal opening 34a, 36a of channels 34, 36. Note as in the embodiment of FIGS. 1-5, the electrodes 42 have a curved configuration and assume this curved configuration when exposed from the sleeve 33. The endoscope 10 is shown within the endoscope receiving channel 38 of the sleeve 33 and has an imaging lens 12 and lighting system 14, and can be a steerable endoscope as described above. The system is used in a similar manner as the system of FIGS. 1-5, with the endoscope 10 inserted into the channel 38 of the sleeve 33 and the system inserted into the patient's body, and the sliders 44 advanced to advance (deploy) the needle electrodes 42 so penetrating tips 42a can penetrate tissue and energy can be applied to the electrodes 42 to treat tissue. Note a stop or markings as described herein can be utilized with the system of FIGS. 8-10 to limit depth penetration of the electrodes 42. Although a separate actuator is shown for each electrode, it is also contemplated that a single actuator could be provided to advance and retract both electrodes.

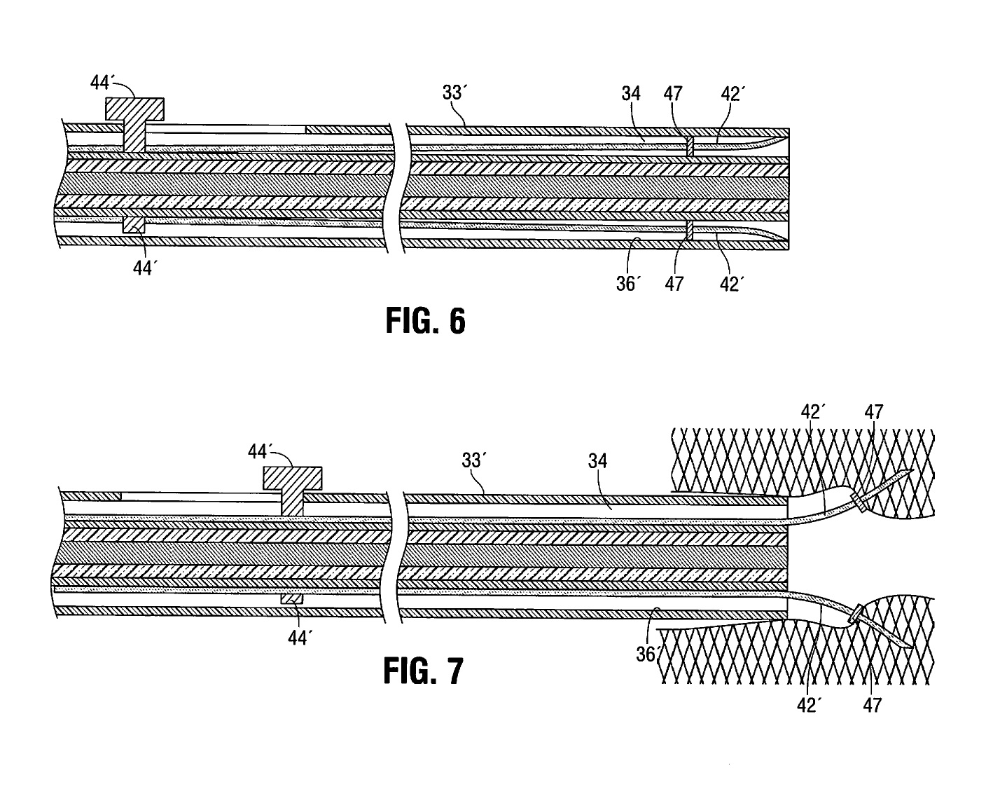

A stop 47 can be positioned circumferentially on an outer surface of the electrode(s) to limit the depth of penetration. This is shown in the embodiment of FIGS. 6 and 7 but the stop can be utilized in any of the other embodiments disclosed herein. The device is otherwise the same as the device of FIG. 8 and therefore corresponding parts are labeled with “prime” designations, e.g., sleeve 33′ electrodes 42′, electrode channel 34′, 36′, endoscope receiving channel 38′ and actuators 44′. When the electrodes 32′ of FIG. 6 are advanced, stop 47 will contact the tissue surface and prevent further penetration of the needle electrode. In this manner, over-insertion of the electrodes is prevented. Prevention of such over-insertion can be utilized in certain embodiments to prevent ablation of tissue. That is, the stop limits penetration to control at the distal end of the device the extent of penetration to provide consistent depth of insertion. Due to the length and flexibility of the device, control of advancement at the distal end is beneficial. By providing consistency, consistent spacing between the electrodes when the device is moved axially to different lesion levels will better ensure consistent spacing. That is, the desired axial distance between lesions will be better maintained as well as the desired radial (circumferential) distance between lesions.

The advantage of the consistent depth penetration of the electrodes 32′ can be appreciated by understanding the spacing for lesion formation. If the needle electrodes are not fully inserted, then undertreatment and/or overtreatment areas will occur. Optimally, when RF energy is applied to the tissue via the electrode tips, the treatment areas are spaced at a minimum of 5 millimeters apart. After application of RF energy, and the device is rotated 45 degrees (or 30 degrees) to provide another application of RF energy, the treatment areas will ideally be equidistantly spaced between the already treated treatment areas. However, if an electrode penetrates a different distance, an inconsistent spacing will occur. Consequently, in the next application of RF energy after device rotation, a treatment region could be closer to another treatment region, and can overlap which can overtreat the tissue and cause undesired tissue ablation. Conversely, a treatment region can be too far from another treatment region which will lead to undertreatment of tissue.

The problem of misalignment and undertreatment/overtreatment is compounded since treatment is in three dimensions. That is, lesions are formed not only in an axial plane but in spaced longitudinal planes, and therefore proper spacing needs to be maintained not only in the axial lesion level, but between axial lesion levels. Therefore, when the device is moved axially to the next axial lesion level and the needle electrodes are deployed, the inconsistent depth of penetration can cause tissue treatment areas too close or too far from other areas between axial planes.

Stated another way, if one electrode terminates more proximally than another electrode due to less insertion, its initial position is improperly rearward of the other electrode. When the electrodes are deployed, the electrode which does not penetrate sufficiently into tissue will not treat the muscle layer but rather treat the mucosal layer when RF energy is applied. When the device is moved to the next lesion level, the problem is compounded as the desired spacing between the treatment areas will not be maintained and RF energy in some regions will be applied too close to the previously treated area causing overheating and unwanted ablation and other regions will be applied too far from the previously treated region causing undertreatment. The effect of such spacing is illustrated in commonly assigned pending patent application Ser. Nos. 14/708,210, filed May 20, 2015, and 14/708,209, filed May 9, 2015 the entire contents of which are incorporated herein by reference.

The stop 34 can also function to provide tactile feedback to the user that the needle electrode 32 has been inserted a desired distance, i.e., to a desired depth, as the user can feel the contact of the stop 34 with the tissue. This prevents under-insertion which can cause undertreatment and the aforedescribed disadvantages.

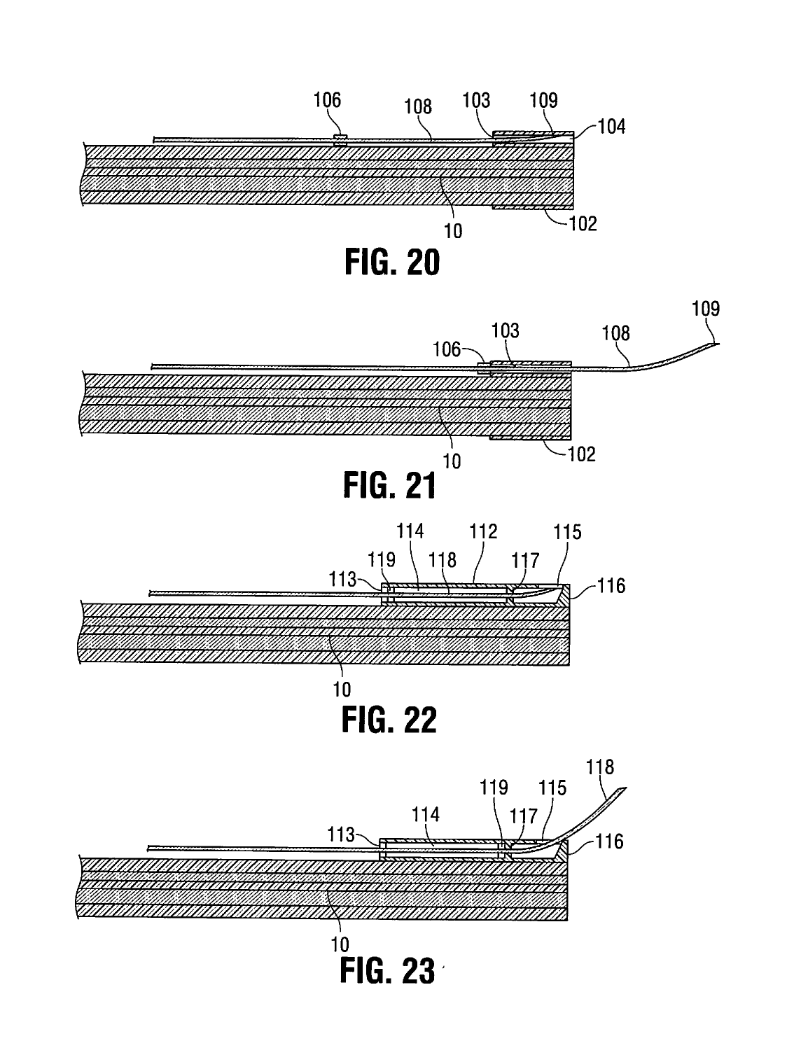

A stop can also be formed on other regions of the electrode such as in an unexposed region of the electrodes as shown for example in FIGS. 20-23 discussed in detail below.

As an alternative to the stop, markings can be provided along the regions of the sleeve to inform the user of the extent of deployment of the needle electrode. This provides another way to control depth penetration of the electrode although it does not provide control at the distal end nor the tactile feedback.

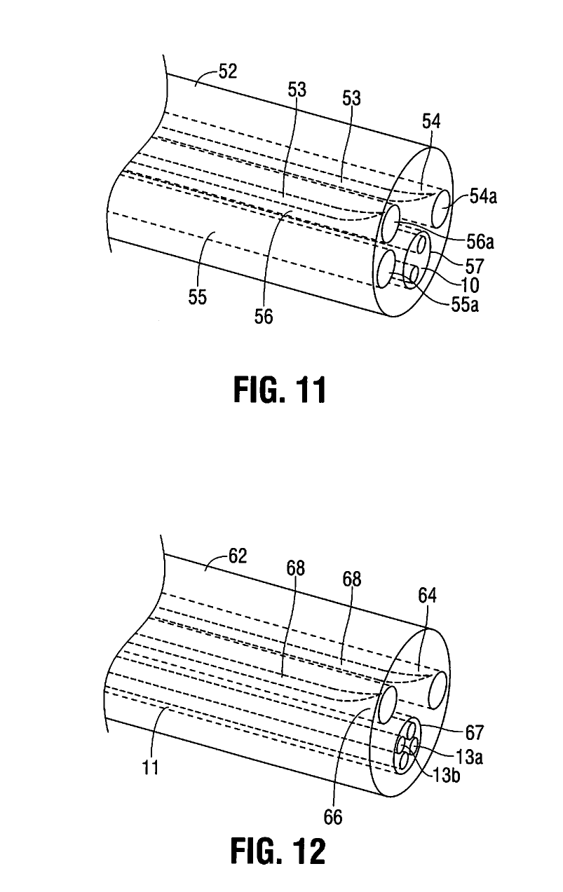

The sleeve of the embodiments disclosed herein can alternatively be provided with a separate channel or lumen for suction and/or irrigation. Such additional channel can be used with the single electrode embodiment of FIGS. 2-5 or the dual electrode embodiments of FIGS. 6-10. FIG. 11 is an example of the sleeve with the dual electrode channel having an additional channel for suction or irrigation. More specifically, the electrodes 53, shown in the retracted position, are advanced through the distal opening 54a, 56a of electrode channels 54, 56, respectively, of sleeve 52 to assume a curved position such as in the electrodes 42 of FIG. 9. The additional channel used for suction and/or irrigation is designated by reference numeral 55 and has a distal opening 55a through which particles can be suctioned and/or fluid can be delivered to the tissue site. Endoscope receiving channel 57 receives endoscope 10. In all other respects, the sleeve 52 is identical in structure and use to sleeve 33 of FIG. 8. In the embodiment of FIG. 12, instead of an additional channel formed in the sleeve, an endoscope 11 with a suction channel 13a and an irrigation channel 13b is utilized. Sleeve 62 is identical to sleeve 33 of FIG. 8, except for the endoscope 11 positioned therein since the endoscope 11 has two additional channels not present in the endoscope of FIG. 10. Thus, sleeve 62 includes electrode channels 64, 66 to receive needle electrodes 68 and an endoscope channel 67. Note the endoscope 11 with the suction/irrigation channel can be inserted in the sleeves of the other embodiments disclosed herein.

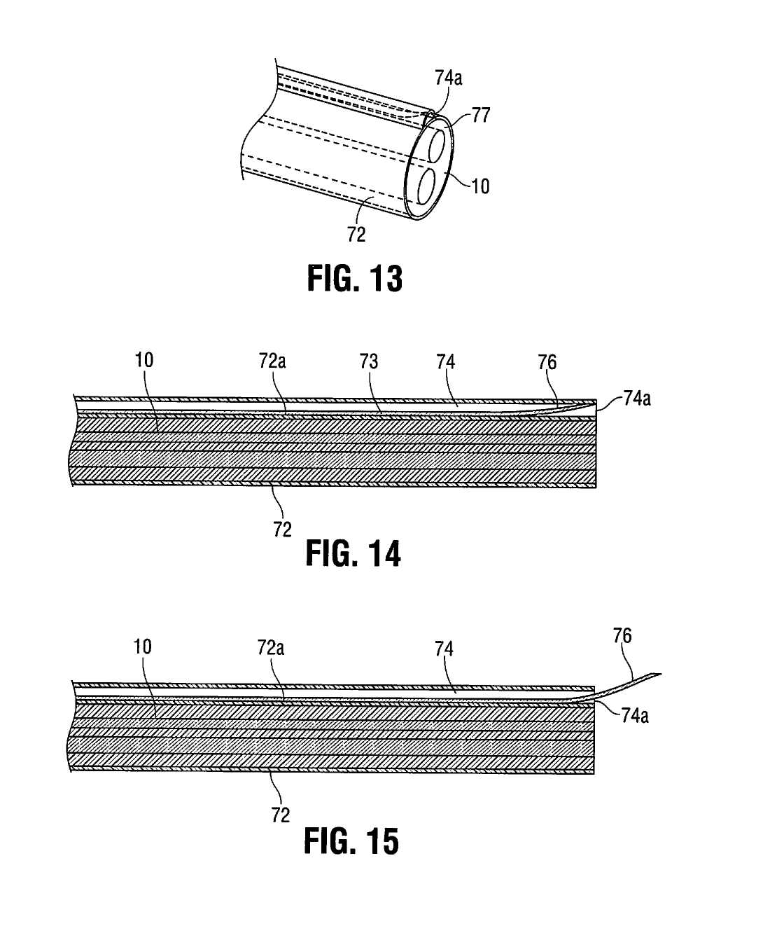

In the previous embodiments, the sleeve was formed with an internal lumen (channel) to slidingly receive the electrodes. In the alternate embodiment of FIGS. 13-15, the sleeve 72 has an electrode channel 74 on an exterior surface 73. Thus, the channel 74 is formed outside wall 72a of sleeve 72. Although one exterior electrode channel 74 is shown, two or more external electrode channels can be provided, extending along a length of the sleeve, and separated by a wall of the sleeve. Needle electrode 76 is slidably received in the channel 74 and is operatively connected to an actuator such as slider 26 of FIG. 2. The actuator moves the electrode 76 from the retracted position of FIG. 14 to the extended position of FIG. 15 to exit distal opening 74a in channel 74. The electrode 76 can be curved as shown and as described above with respect to the other embodiments. The sleeve 72 can include a separate channel for the endoscope or the interior lumen 77 of the sleeve can be dimensioned to receive the endoscope 10.

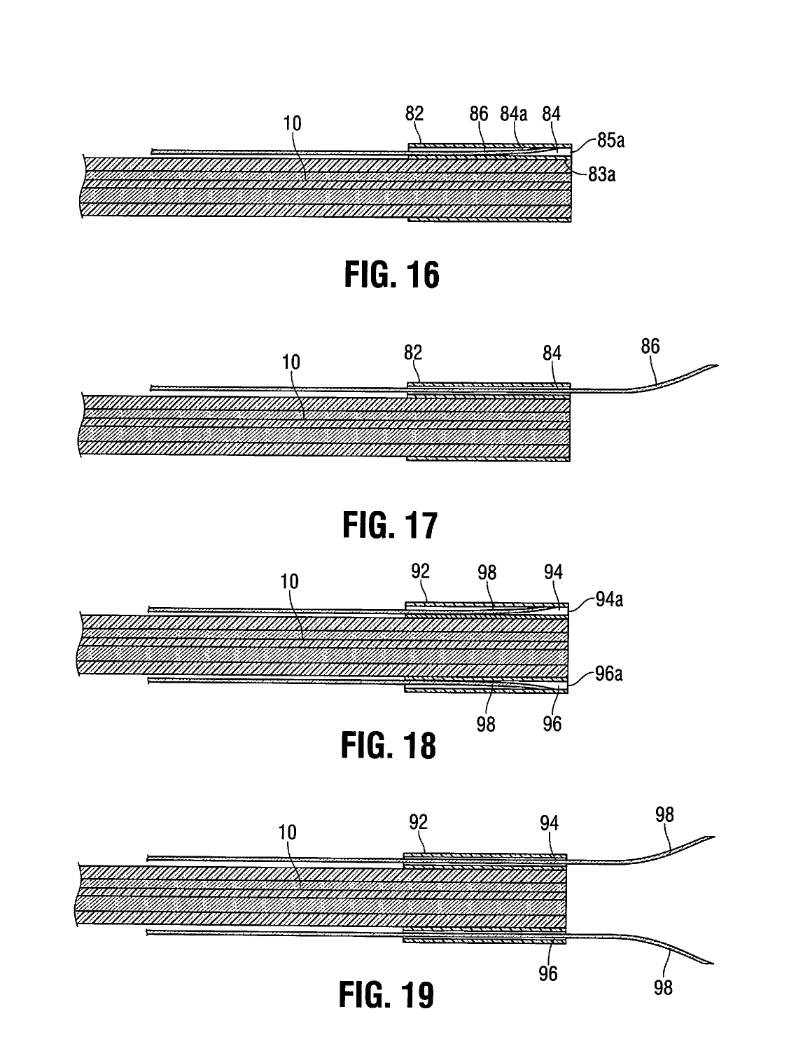

In the embodiments of FIG. 1-15, the sleeve extends the entire length or substantially the entire length of the endoscope. In the alternate embodiments of FIGS. 16-25, the sleeve is of a shorter length and is attachable to only a distal portion of the endoscope. The sleeve 82 of FIG. 16 has an exterior electrode channel 84 to slidingly receive needle electrode 86. Thus, electrode channel 84 is formed between external wall 83a of sleeve 83 and wall 84a of channel 84. An actuator such as that described above moves the electrode 86 from the retracted position of FIG. 16 to the extended position of FIG. 17 to exit distal opening 85a of electrode channel 84 and assume a curved configuration. As can be seen, the electrode or electrode advancer to which the electrode is attached extends outside the sleeve 82 adjacent an exterior surface thereof. This shorter sleeve facilitates insertion of the endoscope 10 through the endoscope receiving channel in the sleeve. The sleeve 92 of FIGS. 18 and 19 is identical to sleeve 82 of FIG. 16 except that two external channels 94, 96 are provided, each slidingly receiving a needle electrode 98. An actuator of the types described above is operatively connected to each of the electrodes 98 for moving the electrodes 98 from the retracted position of FIG. 18 where the distal tip is retained within the sheath 92 and an extended position wherein the distal tip extends from the sleeve 92 out the distal openings 94a, 96a of the electrodes channels 94, 96, respectively, and assumes a curved position. It is also contemplated that as in the other multiple electrode embodiments disclosed herein, a single actuator can be utilized to advance both electrodes.

An alternate embodiment of a stop to limit depth penetration of the electrodes is shown in FIGS. 20 and 21. Stop 106 is fixedly positioned on needle electrode 108, proximal of the sleeve 102, and the electrode tip 109 is contained within electrode channel 104 of sleeve 102. The stop 106, being positioned proximal of sleeve 102, contacts a proximal edge 103 of sleeve 102 to limit the distal advancement of needle electrode 108 as shown in FIG. 21. Thus, when the actuator such as a slider as described above advances the needle electrode 108 from within the confines of sleeve 102, the stop 106 will contact proximal edge 103 and further distal travel of the needle electrode 108 is prevented. This prevents over-penetration of the needle electrode 108 with the aforedescribed attendant advantages. The sleeve 102 also has an endoscope receiving channel to receive endoscope 10 for illumination and imaging. More than one needle electrode can be provided by providing additional electrode channels in the sleeve, and the electrodes can be independently advanceable by individual actuators or advanced together by a single actuator.

The needle electrode(s) can be deployed from the distal opening of the channel as in the aforedescribed embodiments. However, it is also contemplated that alternatively, the electrode(s) can be advanced through a sidewall in the sleeve. This is shown for example in the embodiment of FIGS. 22 and 23, it being understood that such side exit opening can be provided in the other embodiments described herein. Sleeve 112 of FIGS. 22 and 23 has a side opening 115 proximal of the distalmost end 116 which is closed. Needle electrode 118 is slidably received in electrode channel 114 of sleeve 112, the side opening 115 communicating with the electrode channel 114. A stop 119 is fixedly positioned on needle electrode 118 within electrode channel 114 of sleeve 112. Upon advancement of the needle electrode 118 by an actuator such as the slider described herein, stop 119 contacts wall or rib 117 extending radially inwardly from an inner wall of the sleeve 112 to limit depth penetration of the needle electrode 118 to provide the advantages described above. Note the electrode 118 extends out of side opening 115 as shown in FIG. 23 as the tip is advanced and redirected by the inner wall of closed end 116. The proximal wall 113 of the sleeve 112 maintains the stop 119 within the electrode channel 114 and thus provides a limitation on proximal movement of the electrode 118. Stop 117 limits distal movement. The sleeve 112 includes an endoscope receiving channel to receive endoscope 10. Note that more than one needle electrode 118 can be provided by providing additional electrode channels in the sleeve, and the electrodes can be independently advanceable by individual actuators or advanced together by a single actuator.

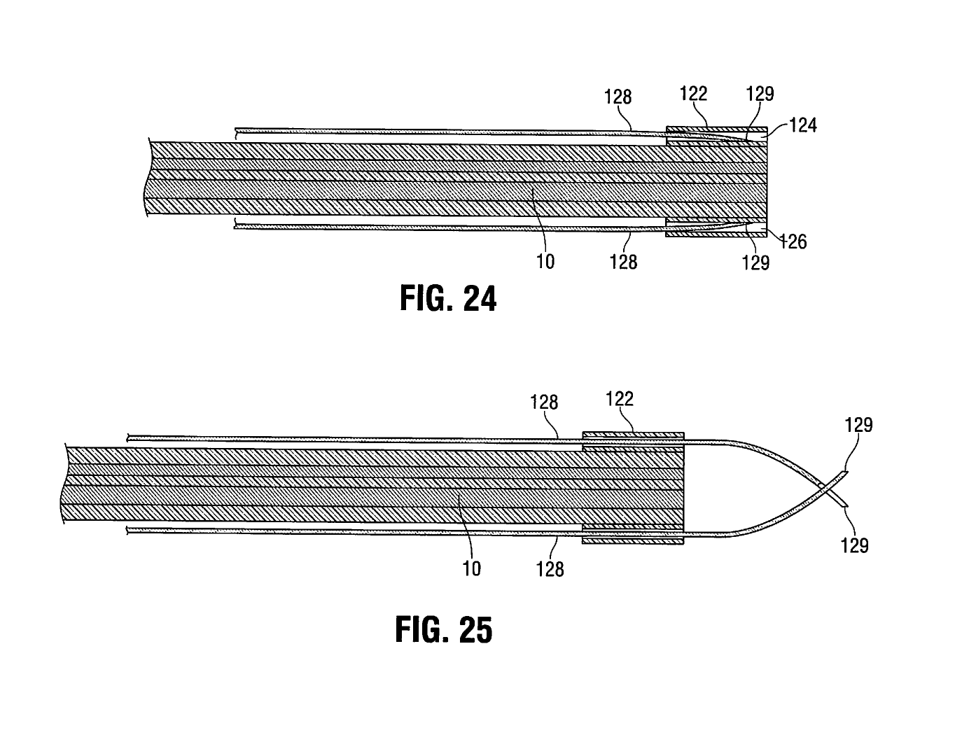

In the embodiment of FIGS. 24 and 25, the needle electrodes 128 when deployed are directed toward each other and then cross over. This enables the distal portions of the electrodes 128 to be visualized. The electrodes 128 are slidably received in a respective electrode channel 124, 126 of sleeve 122 and advanced by an actuator(s) such as a slider described above. An endoscope 10 is positioned within an endoscope channel of sleeve 122. Initially, the needle electrodes 128 are advanced so that their penetrating tips 129 are viewable by the endoscope 10 as shown in FIG. 25. Then the needle electrodes 128 are further advanced so the penetrating tips 129 penetrate tissue as they continue to cross in opposing directions. A stop can be provided as in the various embodiments described above to limit penetration. Alternatively, the sleeve 122 can be of a greater length to extend along a greater length of the endoscope 10.

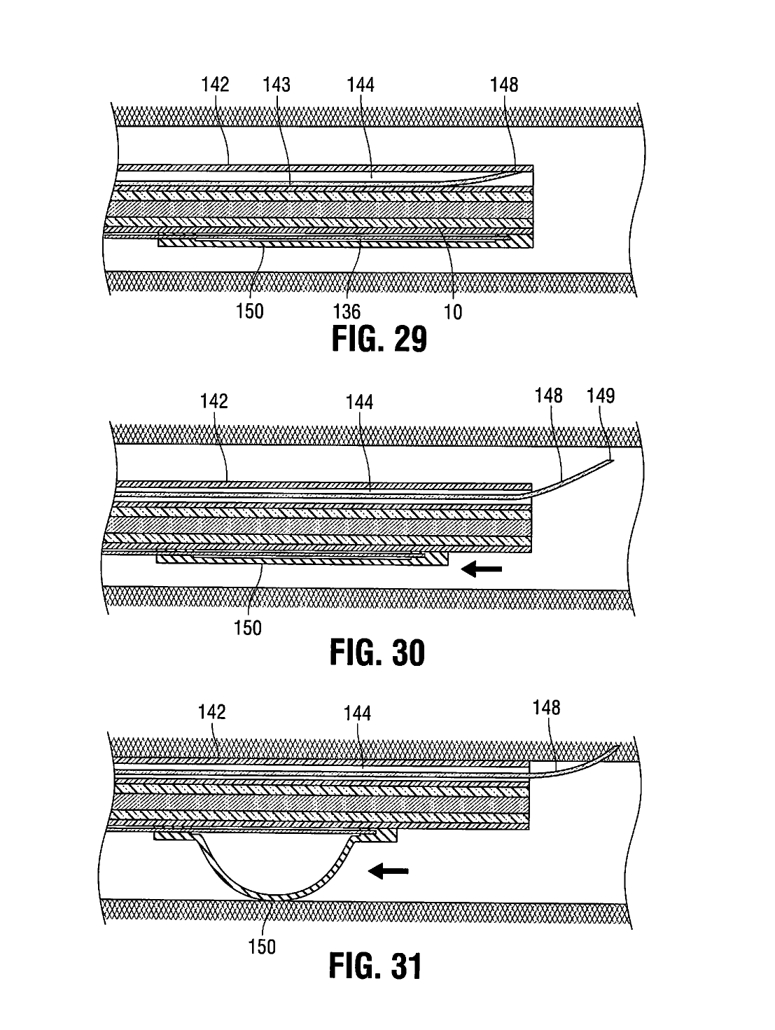

FIGS. 26-31 illustrate mechanisms for directing the electrode needle tips into tissue. As explained above, an articulating or steerable endoscope can be positioned within the sleeve so that articulation or steering of the endoscope tip to an angle with respect to its longitudinal axis, articulates or steers the sleeve which further angles the electrode into tissue. As an alternative, a mechanism can be provided which forces the sleeve toward the tissue wall to direct the penetrating tip of the needle electrode initially or further into the wall. In one embodiment, a balloon is expandable to direct the sleeve (FIGS. 26-28); in an alternate embodiment, a mechanical strut or arm is expanded to direct the sleeve (FIGS. 29-31). Although a single electrode is shown, multiple electrodes are also contemplated.

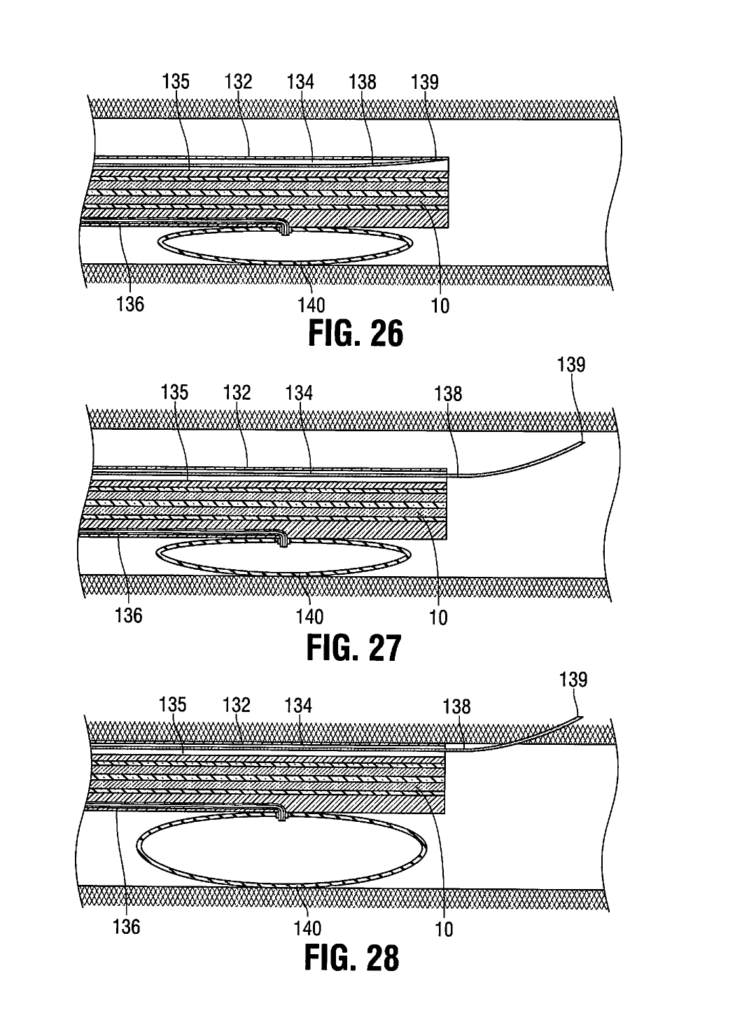

More specifically, in the embodiment of FIGS. 26-28, sleeve 132 has an electrode channel 134, an endoscope channel 135 to receive endoscope 10 and a balloon inflation channel 136 to deliver inflation fluid to inflate balloon 140. A needle electrode 138 is slidingly received in electrode channel 134 and is movable by an actuator such as slider 26 described above from a retracted position of FIG. 26 where penetrating tip 139 is retracted within sleeve 132 and an advanced position of FIG. 27 where the penetrating tip 139 is exposed from the confines of sleeve 132. When desired to direct the needle electrode 138 into tissue or further into tissue if it is already penetrated tissue upon advancement from sleeve 132, the balloon 140 is inflated, thereby forcing the device laterally to the position of FIG. 28 which thereby moves the penetrating tip 139 through the tissue wall as shown.

In the alternate embodiment of FIGS. 29-31, instead of a balloon, a mechanical expander moves the device laterally. More specifically, sleeve 142 has an electrode channel 144, an endoscope channel 143 to receive endoscope 10 and a channel to receive an actuating member such as a cable or wire 136 to expand strut or arm 150. A needle electrode 148 is slidingly received in electrode channel 144 and is movable by an actuator such as slider 26 described above from a retracted position of FIG. 29 where penetrating tip 149 is retracted within sleeve 142 and an advanced position of FIG. 30 where the penetrating tip is exposed. When desired to direct the needle electrode 148 into tissue, or further into tissue if it already penetrated tissue upon advancement from sleeve 142, the actuating member 136 which is attached to the arm 150 is pulled proximally, thereby forcing the device laterally to the position of FIG. 31 which thereby moves the penetrating tip 149 through the tissue wall as shown.

The electrodes, in the various embodiments disclosed herein, have sufficient distal sharpness and strength, when extended, to penetrate a desired depth into the smooth muscle of the lower esophageal sphincter or the cardia of the stomach. The desired depth can range from about 3 mm to about 10 mm, and more preferably between about 5 mm to about 8 mm, although other depth ranges are also contemplated.

In the foregoing embodiments, the sleeve is mounted over the endoscope and the endoscope and sleeve are together inserted into the body. However, it is also contemplated that alternatively the sleeve can be inserted over the endoscope after the endoscope is inserted into the body.

The electrodes are formed of material that conducts radio frequency energy, such as by way of example nickel titanium, stainless steel, e.g., 304 stainless steel, or a combination of nickel titanium and stainless steel.

An electrical insulating material can be coated about the proximal end of each electrode so that when the distal end of the electrode penetrating the smooth muscle of the esophageal sphincter or cardia transmits radio frequency energy, the material insulates the mucosal surface of the esophagus or cardia from direct exposure to the radio frequency energy. Thermal damage to the mucosal surface is thereby avoided. The mucosal surface can also be actively cooled during application of radio frequency energy to further protect the mucosal surface from thermal damage.

The controller can condition the electrodes to operate in a monopolar mode. In this mode, each electrode serves as a transmitter of energy, and an indifferent patch electrode (described later) serves as a common return for all electrodes. In the embodiments with multiple electrodes, the controller can condition the electrodes to operate in a monopolar mode or in a bipolar mode. In the bipolar mode, one of the electrodes comprises the transmitter and another electrode comprises the return for the transmitted energy.

The needle electrodes in use can be maintained in axial (longitudinal) alignment and of controlled tissue penetration due to the presence of the aforedescribed stops.

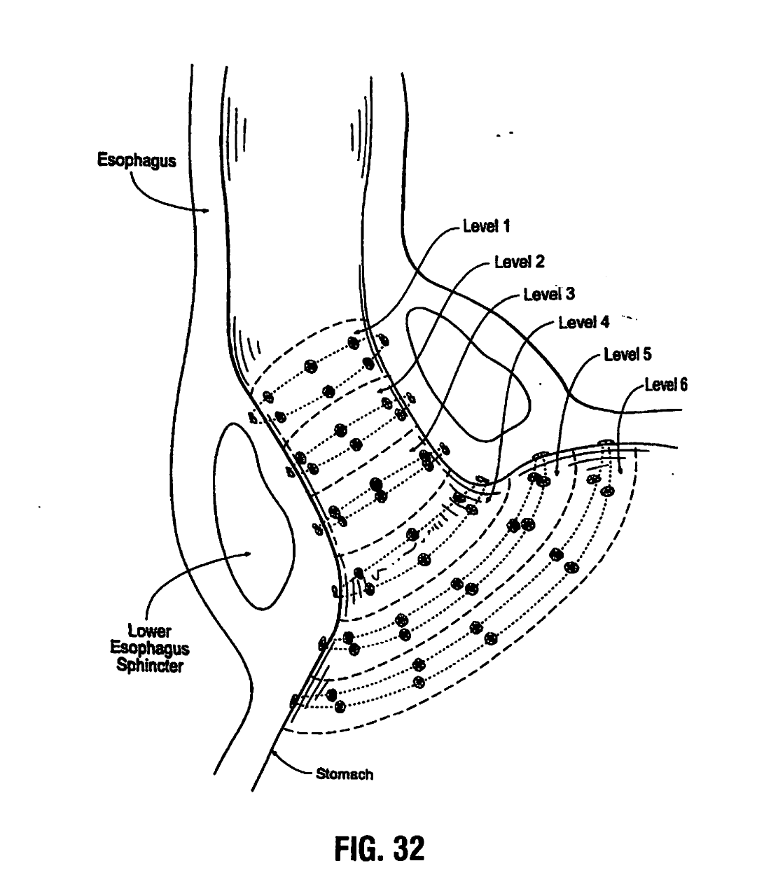

In use of the system, the device is manipulated to create a preferred pattern of multiple lesions comprising circumferential rings of lesions at several axially spaced-apart levels (about 5 mm apart), each level comprising from 8 to 12 lesions. A representative embodiment of the lesion pattern is shown in FIG. 32. The rings are preferably formed in the esophagus in regions above the stomach, at or near the lower esophageal sphincter, and/or in the cardia of the stomach. The rings in the cardia are concentrically spaced about the opening funnel of the cardia. At or near the lower esophageal sphincter, the rings are axially spaced along the esophagus. The sleeve and endoscope are inserted as a unit with the electrode(s) retracted. The needle electrode(s) are then advanced into tissue as shown for application of energy. They can be directed further into tissue by steering of the endoscope or other methods described herein.

Multiple lesion patterns can be created by successive extension and retraction of the electrode(s) accompanied by rotation and/or axial movement of the device. The physician can create a given ring pattern by extending (advancing) the electrode(s) and subsequently retracting the electrodes(s), rotating the sleeve relative to the endoscope or rotating the sleeve and endoscope together, and re-extending the electrode(s) at the targeted treatment site to form a first set of lesions, e.g., four lesions. The physician can rotate the sleeve or sleeve and endoscope by a desired amount, e.g., 30-degrees or 45-degrees, depending upon the number of total lesions desired within 360-degrees. The physician can repeat this sequence until a desired number of lesions within the 360-degree extent of the ring is formed. Additional lesions can be created at different levels by repeatedly advancing the operative element axially, then extending the electrode(s), retracting the electrode(s), rotating the sleeve or sleeve and endoscope and re-extending the electrode(s). This is performed under visualization by the endoscope over which the sleeves of the present invention are mounted.

As shown in FIG. 32, one desirable pattern comprises an axially spaced pattern of six circumferential lesions numbered Level 1 to Level 6 in an inferior direction, with some layers in the cardia of the stomach, and others in the esophagus above the stomach at or near the lower esophageal sphincter. In the embodiment shown, in the Levels 1, 2, 3, and 4, there are eight lesions circumferentially spaced 45-degrees apart. In the Levels 5 and 6, there are twelve lesions circumferentially spaced 30-degrees apart. Clearly, other lesion patterns and other number of lesions and lesion levels can be achieved by the systems herein if desired.

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure as defined by the claims appended hereto.

What is claimed is:

1. A flexible sleeve mountable to an endoscope, the sleeve comprising a proximal portion, a distal portion, a first channel dimensioned to receive an endoscope, and a second channel dimensioned to receive an electrode, the electrode slidable within the second channel from a retracted position wherein a tip of the electrode is unexposed from the sleeve and an extended position wherein the tip of the electrode is exposed from the sleeve and curves toward target tissue.

2. The flexible sleeve of claim 1, wherein the tip of the electrode is a penetrating tip to penetrating the target tissue.

3. The flexible sleeve of claim 1, wherein the flexible sleeve includes a third channel and a second electrode slidable within the third channel from a retracted position wherein a tip of the second electrode is unexposed from the sleeve and an extended position wherein the tip of the second electrode is exposed from the sleeve and curves toward target tissue.

4. The flexible sleeve of claim 1, further comprising an actuator operatively connected to the electrode for moving the electrode between retracted and exposed positions.

5. The flexible sleeve of claim 2, further comprising a stop positioned on the electrode to control a depth of penetration of the electrode to provide a consistent depth of penetration at the lesion levels.

6. The flexible sleeve of claim 1, further comprising an aspiration lumen for aspiration of particles.

7. The flexible sleeve of claim 1, wherein the second channel is on an exterior surface of the sleeve.

8. The flexible sleeve of claim 1, wherein the sleeve includes an expandable member, the expandable member expandable to force the electrode further into tissue.

9. The flexible sleeve of claim 8, wherein the expandable member is an inflatable balloon.

10. A system comprising a flexible endoscope and a flexible sleeve mountable over the endoscope to apply energy to form lesions in a treatment of gastrointestinal reflux disease under visualization, the sleeve having a first channel to receive the endoscope therein and a second channel for slidably receiving an electrode, the electrode slidable within the second channel from a retracted position wherein a tip of the electrode is unexposed from the sleeve and an extended position wherein the tip of the electrode is exposed from the sleeve and curves toward target tissue, the electrode connectable to a generator for application of energy to tissue to form lesions to tighten a sphincter.

11. A system of claim 10, wherein non-ablative energy is applied to the electrode.

12. A system of claim 10, wherein the electrode includes a stop to limit penetration depth to prevent overtreatment of tissue.

13. A system of claim 10, wherein the sleeve is placed over a substantial portion of the endoscope.

14. A system of claim 10, wherein the sleeve is placed over only a distal portion of the endoscope.

15. A system of claim 10, wherein the sleeve includes a second channel and a second electrode slidable within the second channel from a retracted position wherein a tip of the second electrode is unexposed from the sleeve and an extended position wherein the tip of the second electrode is exposed from the sleeve and curves toward target tissue, the second electrode connectable to a generator for application of energy to tissue to form lesions to tighten a sphincter.

16. A method for forming lesions in a sphincter comprising:
a. providing an endoscope;
b. providing a flexible sleeve;
c. mounting the flexible sleeve over at least a distal portion of the endoscope;
d. inserting the sleeve and endoscope into aa body to position adjacent target tissue;
e. extending an electrode from the sleeve to penetrate tissue; and
f. applying energy to tissue to form lesions.

17. The method of claim 16, wherein the step of applying energy applies non-ablative energy.

18. The method of claim 16, wherein the electrode includes a stop and the step of extending the electrode extends the electrode until the stop prevents further penetration to thereby limit the depth of penetration to control application of energy.

19. The method of claim 16, further comprising the step of expanding an expandable member to advance the electrode further into tissue.
